(19) United States
(12) Patent Application Publication
(10) Pub. No.: US 2014/0257407 A1
(43) Pub. Date: Sep. 11, 2014

(54) FIXATION DEVICE FOR QUADRILATERAL PLATE FRACTURES OF THE ACETABULUM VIA ILIOINGUINAL APPROACH

(71) Applicant: Wuhan General Hospital of Guangzhou Military, Wuhan City (CN)

(72) Inventors: Xianhua CAI, Wuhan City (CN); Ximing LIU, Wuhan City (CN)

(73) Assignee: Wuhan General Hospital of Guangzhou Military, Wuhan City (CN)

(21) Appl. No.: 14/180,235

(22) Filed: Feb. 13, 2014

(30) Foreign Application Priority Data
Mar. 8, 2013 (CN) 201320106378.0

(51) Int. Cl. A61B 17/80

(52) U.S. Cl. CPC A61B 17/8066; A61B 17/8033
USPC 606/298

(57) ABSTRACT

The present invention relates to an orthopedic internal fixator, especially a fixator for quadrilateral plate fractures of the acetabulum via ilioinguinal approach, which includes a shaped fixing titanium plate, whose outer contour is arc-shaped and basically the same as the pelvic arcuate line. While the pre-bending curvature of the proximal plate for quadrilateral plate is slightly greater than that of the arcuate line, making the proximal end of the titanium plate slightly away from the arcuate line. Elliptical fixing holes are evenly arranged along the fixing titanium plate, while the 3˜5 holes on the middle part are low-profile oval shape fixing holes. The titanium plate in the quadrilateral plate presents as a tilted slope with the exterior side being higher than the interior side, whose gradient is less than that of the bone surface of a linea terminalis of pelvis by about 15 degrees. The tilted direction of the two ends of the fixing titanium plate is opposite to that of the middle part, and is presenting as inside-outside gradually increasing tilted slopes with the exterior sides being lower than the interior sides and their gradients are about 15 degrees, thus the shaped fixing titanium plate placed behind the bone surface of a linea terminalis of pelvis with two ends upwarping. The invention provides a device for the treatment of quadrilateral plate fractures of the acetabulum via ilioinguinal approach, which has strong internal fixation capability and is easy to use.

### FIELD OF THE INVENTION

The invention relates to an orthopedic internal fixator, exclusively used in the treatment of various quadrilateral plate fractures of the acetabulum via ilioinguinal approach, which has an internal fixation system of instantly reduction and local progressive dynamic compression mechanism for the fractures of the quadrilateral plate.

### DESCRIPTION OF THE PRIOR ART

With the increase in traffic injury and industrial accidents, the incidence of acetabular fractures increases every year. And acetabular fractures are currently accounting for 1%-3% of the total fracture cases with a fatality rate of about 5%-20% and a disability rate up to 50%-60%. And the quadrilateral plate of acetabulum is frequently involved in acetabular fractures. Current studies suggest that the curative effect of operative treatment is superior to conservative treatment. However, the anatomical location of the quadrilateral plate of acetabulum is deep, and the bone is thin, surrounded by vital blood vessels, nerves and is close to organs. Thus, direct internal fixation is difficult. The combined anterior and posterior approach internal fixation is the most commonly used, with the holes of anterior reconstruction titanium plate placing at the quadrilateral plate having screw holes unoccupied. Some surgeons recommend static fixation, which is difficult to achieve satisfactory fixation. Therefore, the internal fixation treatment of quadrilateral plate fractures of the acetabulum is a major challenge in traumatic orthopedics.

### SUMMARY OF THE INVENTION

The invention aims to overcome the drawbacks of the prior art to provide a fixator for quadrilateral plate fractures of the acetabulum via ilioinguinal approach, which has high internal fixation capability and is easy to use.

To solve the above technical problems, one of the technical solutions of the invention is to provide a fixator for quadrilateral plate fractures of the acetabulum via ilioinguinal approach, which is distinguished at including a shaped fixing titanium plate. A plurality of fixing holes are evenly arranged on the fixing titanium plate, the outer contour of the plate body of the fixing titanium plate is arc-shaped which is basically the same as the pelvic arcuate line. The pre-bending curvature of the middle part of the fixation titanium plate is greater than the arcuate line. And the middle part of the fixation titanium plate presents as a tilted slope with the exterior side being higher than the interior side. The gradient of the middle part of the fixation titanium plate is less than that of the bone surface of a linea terminalis of pelvis. The tilted direction of two ends of the fixing titanium plate is opposite to that of the middle part of the fixing titanium plate, and is present as inside-outside gradually increasing tilted slopes with the exterior sides being lower than the interior sides. The gradients of the two ends of the fixing titanium plate are greater than that of the bone surface of the linea terminalis of pelvis. The shaped fixing titanium plate is placed behind the bone surface of the linea terminalis of pelvis with two ends upwarping.

Further, after the proximal and the distal fixing screws are embedded into the fixing holes successively, the proximal end and the distal end, whose angles are partly twisted, will produce torque force to the screws of the middle part of the quadrilateral plate due to its elastic restoring force, thus fixing the fractures of the quadrilateral plate.

Further, the fixing holes on the two ends of the fixing titanium plate are elliptical; the fixing holes on the middle part of the fixing titanium plate are low-profile oval shape; and the fixing titanium plate is fixed on the bones by fixing screws going through the fixing holes.

Further, the gradient of the middle part of the fixing titanium plate is 14-16 degrees less than that of the linea terminalis of pelvis bone surface.

Further, the gradients of the two ends of the fixing titanium plate are 14-16 degrees greater than that of the linea terminalis of pelvis bone surface.

Further, each of the two ends of the fixing titanium plate has at least 3 fixing holes.

Further, the middle part of the fixing titanium plate has 3-5 fixing holes.

Compared with the prior art, the invention possesses advantages as follows:

1) As a fixator for quadrilateral plate fractures of the acetabulum via ilioinguinal approach, the approach (ilioinguinal approach) is commonly used. When the proximal and the distal fixing screws are embedded into the fixing holes successively, the proximal end and the distal end, whose angles are partly twisted, will produce torque force to the screws of the middle part of the quadrilateral plate due to its elastic restoring force. After the internal fixation is accomplished, the torque force will produce dynamical stabilization effect to the fracture blocks of the quadrilateral plate.

2) As a fixator for quadrilateral plate fractures of the acetabulum via ilioinguinal approach, the reduction fixation is performed visually to the fracture blocks of the quadrilateral plate, avoiding the risk of joint penetration by the screws in the quadrilateral plate.

3) As a fixator for quadrilateral plate fractures of the acetabulum via ilioinguinal approach, it possesses not only the effect of blocking but also some effect of pulling (the bone of the quadrilateral plate is mainly cancellous bone with plenty blood supply, which is easily to form callus, and will in turn partly bury the exposed screws and is almost equivalent to inner fixation in bone) to the fracture blocks of the quadrilateral plate.

4) As a fixator for quadrilateral plate fractures of the acetabulum via ilioinguinal approach, it has certain fixation effect on the comminuted fracture fragments of quadrilateral plate.

5) As a fixator for quadrilateral plate fractures of the acetabulum via ilioinguinal approach, the internal fixation material is titanium alloy, which will not influence the MRI examination postoperatively.

### BRIEF DESCRIPTION OF THE DRAWINGS

FIG. 1 is a front view of the fixing titanium plate in the embodiment of the invention;

FIG. 2 is a rear view of the fixing titanium plate in the embodiment of the invention;

FIG. 3 is a schematic diagram 1 of the use condition in the embodiment of the invention;

FIG. 4 is a schematic diagram 2 of the use condition in the embodiment of the invention;

Hereinto, 1—fixing titanium plate; 2—fixing holes; 3—fixing screws on the two ends of the fixing titanium plate; 4—fixing screws on the middle part of the fixing titanium plate.

### DETAILED DESCRIPTION OF THE PREFERRED EMBODIMENTS

The invention will be described in further detail through embodiments combined with figures as follows:

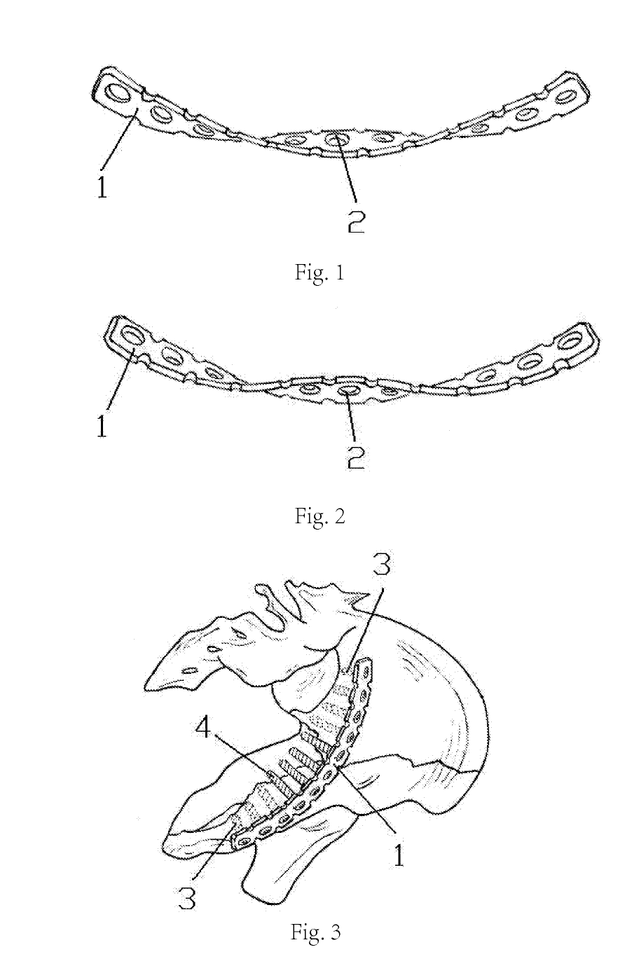

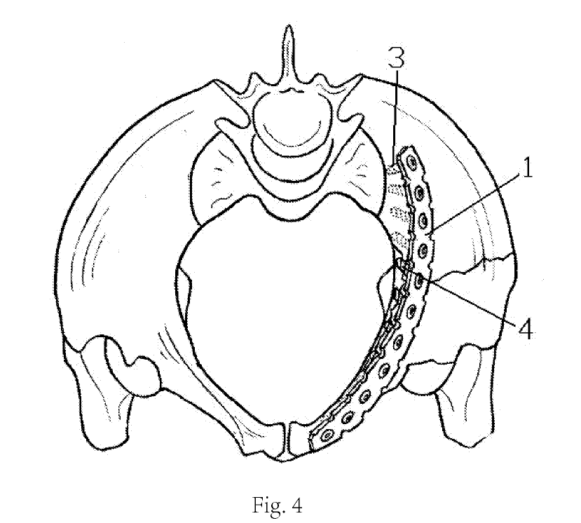

Please refer to FIG. 1, FIG. 2, FIG. 3 and FIG. 4. The embodiment of a fixator for quadrilateral plate fractures of the acetabulum via ilioinguinal approach comprises a shaped titanium plate 1. The outer contour of the plate body of the shaped titanium plate 1 is arc-shaped and is basically the same as the pelvic arcuate line. The pre-bending curvature of the middle part of the fixing titanium plate 1 is greater than the arcuate line, which makes the proximal side of the titanium plate slightly away from the arcuate line. And the middle part of the fixing titanium plate 1 presents as a tilted slope with the exterior side being higher than the interior side, whose gradient is less than that of the bone surface of a linea terminalis of pelvis. The tilted direction of the two ends of the fixing titanium plate 1 is opposite to that of the middle part, and is present as inside-outside gradually increasing tilted slopes with the exterior sides being lower than the interior sides. The shaped fixing titanium plate 1 is placed behind the bone surface of the linea terminalis of pelvis with two ends upwarping. A plurality of fixing holes 2 are evenly arranged on the fixing titanium plate 1. The fixing holes 2 on the two ends of the fixing titanium plate 1 are elliptical while the fixing holes 2 on the middle part of the fixing titanium plate 1 are low-profile oval shape. The fixing titanium plate 1 is fixed on the bones by the fixing screws going through the fixing holes 2. When the proximal and the distal fixing screws are embedded into the fixing holes successively, the proximal end and the distal end, whose angles are partly twisted, will produce torque force to the screws of the middle part in the quadrilateral plate due to its elastic restoring force, thus fixing the fractures of the quadrilateral plate. As showed in FIG. 4, the wording “interior” and “exterior” mentioned above are with respect to the interior side and exterior side of the pelvic cavity.

Further, the gradient of the middle part of the fixing titanium plate 1 is 14-16 degrees less than that of the linea terminalis of pelvis bone surface.

Further, the gradients of the two ends of the fixing titanium plate are 14-16 degrees greater than that of the linea terminalis of pelvis bone surface.

Further, each of the two ends of the fixing titanium plate 1 has at least 3 fixing holes 2.

Further, the middle part of the fixing titanium plate 1 has 3-5 fixing holes 2.

Further, the fixing screws 3 on the two ends of the fixing titanium plate are 3.5 mm-diameter full-thread cancellous bone screws, while the fixing screws 4 for the middle part of the fixing titanium plate are 3.5 mm-diameter cortical bone screws. The fixing screws 3 on the two ends of the fixing titanium plate and the fixing screws 4 on the middle part of the fixing titanium plate are 28-65 mm in length.

Referring to FIG. 3 and FIG. 4, the solid line screws in the embodiment of the invention are the fixing screws 4 on the middle part of the fixing titanium plate, which are partly exposed to the outside of the fracture surface. The dotted line screws are the fixing screws 3 on the two ends of the fixing titanium plate. When the embodiment of the invention is applied, the fixing titanium plate 1 moves towards the pelvic cavity to ½-⅓ in diameter of the screw holes to expose the fixing titanium plate 1 to outside of the inner edge of the linea terminalis of pelvis. The fixing titanium plate 1 matches perfectly with the linea terminalis of pelvis after installation.

Further, the fixing holes (no less than 3 in number) on both ends of the fixing titanium plate in the embodiment of the invention are all elliptical, through which the fixing titanium plate is fixed along the bone slightly away from the arcuate line and to the superior ramus of pubis bone by fixing screws.

Further, the fixing holes (3-5 in number) on the middle part of the fixing titanium plate in the embodiment of the invention are low-profile oval shape, through which the fixing titanium plate is fixed to the fracture blocks of the quadrilateral plate by the fixing screws with ⅓-½ of the screw bodies exposed to the outside of the bones (solid screws in FIG. 3 and FIG. 4).

Further, the fixing screws on the two ends of the fixing titanium plate in the embodiment of the invention are 3.5 mm-diameter full-thread cancellous bone screws, while the fixing screws on the middle part of the fixing titanium plate are 3.5 mm-diameter cortical bone screws. The fixing screws on the two ends of the fixing titanium plate and the fixing screws on the middle part of the fixing titanium plate are all 28-65 mm in length. After the fixing screws for arcuate line bone and superior ramus of pubis bone are sequentially implanted, the partly “tiwisted” fixing titanium plate at arcuate line bone and superior ramus of pubis bone then tightly contacts with the bone surface of the linea terminalis of pelvis and produces strong torque force to the fixing screws on the middle part of the fixing titanium plate (solid screws in FIG. 3 and FIG. 4), which can effectively maintain the reduction of fracture blocks of the quadrilateral plate and prevent the shift of these fracture blocks towards the pelvis cavity. Those screws with ⅓-½ of the bodies screwed into the bone surface of the quadrilateral plate possess the functions of preventing separation of fracture blocks and direct fracture fixing. After fixation, those screws on the middle part of the fixing titanium plate, which are partly imbedded in the bone, have no left or right shift.

Further, referring to FIGS. 1, 2, 3 and 4, on the two ends of the titanium plate in the embodiment of the invention, no less than 2 of the 3.5 mm cortical screws are used to fix the titanium plate so as to stabilize the partial fractures of anterior column or through anterior column. Holes are drilled through the screw holes on the titanium plate and parallel to the quadrilateral plate surface. And 3-5 of the 3.5 mm cortical screws, i.e. the fixing screws 4 on the middle part of the titanium plate, are screwed into the drilled holes. The length of the screws should surpass the distal end of the fracture by at least 10 mm The fixing screws partly exposed to the outside of the quadrilateral plate surface can resist and pull up the fracture blocks, and maintain the reduction of fracture.

Above is a further detailed description of the invention combined with the specific the embodiments and is not a limit to the invention. It will be understood by those skilled in the art that various simple deduction or replacement within the conception of the invention can be made therein without departing from the spirit and scope of the invention, which should be regarded as belonging to the protection scope of the invention

What is claimed is:

1. A fixator for quadrilateral plate fractures of the acetabulum via ilioinguinal approach, comprising a shaped fixing titanium plate, a plurality of fixing holes are evenly arranged on said fixing titanium plate, a outer contour of a plate body of said fixing titanium plate is arc-shaped and is basically the same as pelvic arcuate line, a pre-bending curvature of a middle part of the fixing titanium plate is greater than the arcuate line, presenting as a tilted slope with the exterior side being higher than the interior side, whose gradient is less than that of a bone surface of a linea terminalis of pelvis, the tilted direction of two ends of the fixing titanium plate is opposite to that of said middle part, and is presenting as inside-outside gradually increasing tilted slopes with the exterior sides being lower than the interior sides, and the gradients of said two ends of the fixing titanium plate is greater than that of the bone surface of linea terminalis of pelvis, said shaped fixing titanium plate is placed behind the bone surface of the linea terminalis of pelvis with said two ends upwarping.

2. The fixator for quadrilateral plate fractures of the acetabulum via ilioinguinal approach according to claim 1, wherein said fixing holes on said two ends of the fixing titanium plate are elliptical, and said fixing holes on said middle part of the fixing titanium plate are low-profile oval shape, said fixing titanium plate is fixed on the bones by fixing screws going through said fixing holes.

3. The fixator for quadrilateral plate fractures of the acetabulum via ilioinguinal approach according to claim 1, wherein the gradient of said middle part of the fixing titanium plate is 14-16 degrees less than that of linea terminalis of pelvis bone surface.

4. The fixator for quadrilateral plate fractures of the acetabulum via ilioinguinal approach according to claim 1, wherein the gradients of said two ends of the fixing titanium plate are 14-16 degrees greater than that of linea terminalis of pelvis bone surface.

5. The fixator for quadrilateral plate fractures of the acetabulum via ilioinguinal approach according to claim 3, wherein the gradients of said two ends of the fixing titanium plate are 14-16 degrees greater than that of linea terminalis of pelvis bone surface.

6. The fixator for quadrilateral plate fractures of the acetabulum via ilioinguinal approach according to claim 2, wherein the gradient of said middle part of the fixing titanium plate is 14-16 degrees less than that of linea terminalis of pelvis bone surface.

7. The fixator for quadrilateral plate fractures of the acetabulum via ilioinguinal approach according to claim 2, wherein the gradients of said two ends of the fixing titanium plate are 14-16 degrees greater than that of linea terminalis of pelvis bone surface.

8. The fixator for quadrilateral plate fractures of the acetabulum via ilioinguinal approach according to claim 6, wherein the gradients of said two ends of the fixing titanium plate are 14-16 degrees greater than that of linea terminalis of pelvis bone surface.

9. The fixator for quadrilateral plate fractures of the acetabulum via ilioinguinal approach according to claim 1, wherein each of said two ends of the fixing titanium plate has at least 3 fixing holes.

10. The fixator for quadrilateral plate fractures of the acetabulum via ilioinguinal approach according to claim 1, wherein said middle part of the fixing titanium plate has 3-5 fixing holes.

11. The fixator for quadrilateral plate fractures of the acetabulum via ilioinguinal approach according to claim 2, wherein each of said two ends of the fixing titanium plate has at least 3 fixing holes.

12. The fixator for quadrilateral plate fractures of the acetabulum via ilioinguinal approach according to claim 2, wherein said middle part of the fixing titanium plate has 3-5 fixing holes.

13. The fixator for quadrilateral plate fractures of the acetabulum via ilioinguinal approach according to claim 11, wherein said middle part of the fixing titanium plate has 3-5 fixing holes.

14. The fixator for quadrilateral plate fractures of the acetabulum via ilioinguinal approach according to claim 2, wherein the fixing screws on said two ends of the fixing titanium plate are 3.5 mm-diameter full-thread cancellous bone screws, and the length of the fixing screws on said two ends of the fixing titanium plate are 28-65 mm.

15. The fixator for quadrilateral plate fractures of the acetabulum via ilioinguinal approach according to claim 2, wherein the fixing screws on said middle part of the fixing titanium plate are 3.5 mm-diameter cortical bone screws, and the length of the fixing screws on said middle part of said fixing titanium plate are 28-65 mm.

16. The fixator for quadrilateral plate fractures of the acetabulum via ilioinguinal approach according to claim 14, wherein the fixing screws on said middle part of the fixing titanium plate are 3.5 mm-diameter cortical bone screws, and the length of the fixing screws on said middle part of the fixing titanium plate are 28-65 mm.

17. A fixator for quadrilateral plate fractures of the acetabulum via ilioinguinal approach, comprising a shaped fixing titanium plate, a plurality of fixing holes are evenly arranged on said fixing titanium plate, a outer contour of a plate body of said fixing titanium plate is arc-shaped and is basically the same as the pelvic arcuate line, the pre-bending curvature of a middle part of the fixing titanium plate is greater than the arcuate line, presenting as a tilted slope with the exterior side being higher than the interior side, whose gradient is less than that of a bone surface of a linea terminalis of pelvis, the tilted direction of two ends of the fixing titanium plate is opposite to that of said middle part, and is presenting as inside-outside gradually increasing tilted slopes with the exterior sides being lower than the interior sides, and the gradients of said two ends of the fixing titanium plate is greater than that of the bone surface of linea terminalis of pelvis, said shaped fixing titanium plate is placed behind the bone surface of the linea terminalis of pelvis with said two ends upwarping; after the proximal and the distal fixing screws are embedded into said fixing holes successively, the proximal end and the distal end, whose angles are partly twisted, will produce torque force to the screws of said middle part in said quadrilateral plate due to its elastic restoring force, thus fixing the fractures of the quadrilateral plate.

18. The fixator for quadrilateral plate fractures of the acetabulum via ilioinguinal approach according to claim 17, wherein said fixing holes on said two ends of the fixing titanium plate are elliptical, and said fixing holes on said middle part of the fixing titanium plate are low-profile oval shape, said fixing titanium plate is fixed on the bones by the fixing screws going through said fixing holes.

19. The fixator for quadrilateral plate fractures of the acetabulum via ilioinguinal approach according to claim 17, wherein the gradient of said middle part of the fixing titanium plate is 14-16 degrees less than that of linea terminalis of pelvis bone surface.

20. The fixator for quadrilateral plate fractures of the acetabulum via ilioinguinal approach according to claim 19, wherein the gradients of said two ends of the fixing titanium plate are 14-16 degrees greater than that of linea terminalis of pelvis bone surface.
